(19) United States
(12) Plant Patent Application Publication
(10) Pub. No.: US 2003/0014793 P1
(43) Pub. Date: Jan. 16, 2003

(54) LANTANA PLANT NAMED 'ROBPATPAS'

(76) Inventor: Robert J. Roberson, Grain Valley, MO (US)

Correspondence Address: Catherine Anne Whealy, Proprietary Rights International, 1513 Melody Lane, Roanoke, TX, 76262, US

(21) Appl. No.: 09/895,635

(22) Filed: Jun. 30, 2001

(51) Int. Cl. A01H 5/00

(52) U.S. Cl. PLT/227

(57) ABSTRACT

A distinct cultivar of Lantana plant named ‘Robpatpas’, characterized by itsupright and outwardly spreading plant habit; mounded plant form; vigorous growth habit; rapid growth rate; freely flowering habit; and bright pink-colored flowers.

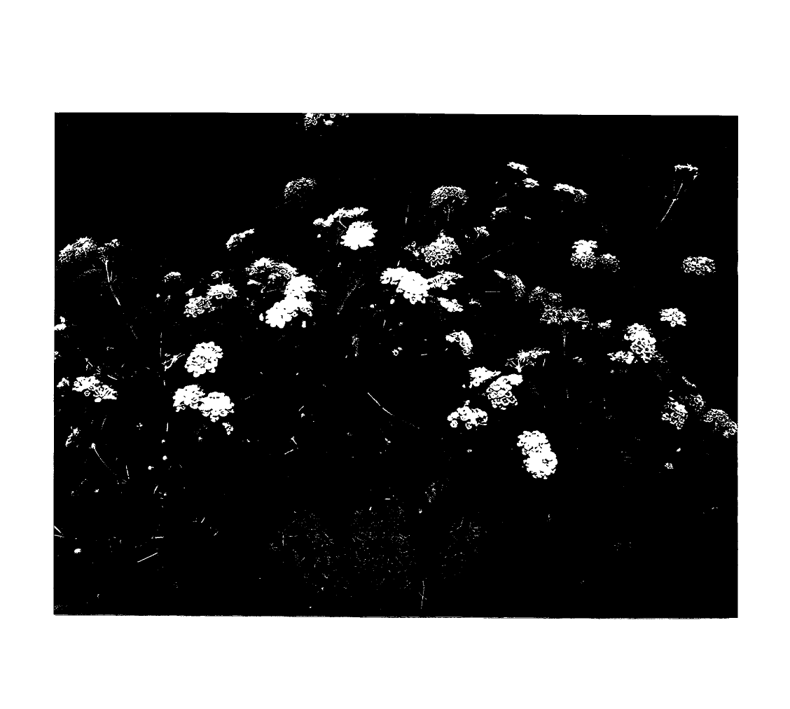

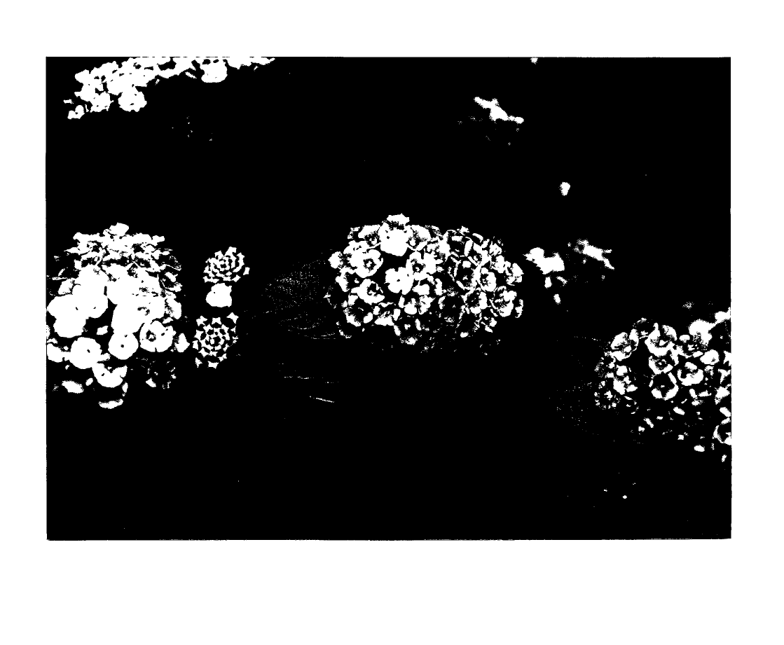

### BACKGROUND OF THE INVENTION

The present Invention relates to a new and distinct cultivar of Lantana plant, botanically known as Lantana camara, and hereinafter referred to by the cultivar name Robpatpas.

The new Lantana is a product of a planned breeding program conducted by the Inventor in Grain Valley, Missouri. The obj ective ofthe breeding program is to create freely-flowering Lantanas with bright pinkcolored flowers.

The new Lantana originated from a cross made by the Inventor during the spring, 1995, of the Lantana cultivar Patriot Hot Country, disclosed in U.S. Plant Patent application serial number 09/350,381, as the female, or seed parent, with an unidentified Lantana selection as the male, or pollen parent. The new Lantana was selected during the winter, 1995, as a single plant from the resulting progeny by the Inventor in a controlled environment in Grain Valley, Missouri, on the basis of its bright pink-colored flowers.

Asexual reproduction ofthe new cultivar by terminal cuttings taken in a controlled environment in Grain Valley, Missouri, has shown that the unique features ofthis new Lantana are stable and reproduced true to type in successive generations.

### SUMMARY OF THE INVENTION

Plants of the cultivar Robpatpas have not been observed under all possible environmental conditions. The phenotype may vary somewhat with variations in environment and culture such as temperature, light intensity, daylength, water status, and/or fertilizer rate or type without, however, any variance in genotype.

The following traits have been repeatedly observed and are determined to be the unique characteristics of ‘Robpatpas’. These characteristics in combination distinguish ‘Robpatpas’ as a new and distinct Lantana cultivar:

1. Upright and outwardly spreading plant habit; mounded plant form.

2. Vigorous growth habit; rapid growth rate.

3. Freely flowering habit.

4. Bright pink-colored flowers.

Plants of the new Lantana are comparable to plants of the female parent, the cultivar Patriot Hot Country. In side-by-side comparisons conducted in Grain Valley, Missouri, plants of the new Lantana have bright pink-colored flowers whereas plants of the cultivar Patriot Hot Country have magenta-colored flowers.

Plants of the new Lantana differ from plants ofthe male parent, the unidentified Lantana selection, and other Lantana cultivars known to the Inventor primarily in the new Lantana's unique combination of mounded plant habit, bright pink flower color, and freely-flowering habit.

### BRIEF DESCRIPTION OF THE PHOTOGRAPHS

The accompanying colored photographs illustrate the overall appearance of the new cultivar, showing the colors as true as it is reasonably possible to obtain in colored reproductions of this type. Colors in the photographs may differ slightly from the color values cited in the detailed botanical description which accurately describe the colors of the new Lantana.

The photograph on the first sheet comprises a side perspective view of typical flowering plants of ‘Robpatpas’ grown in a hanging basket container. The photograph on the second sheet comprises a close-up view of typical inflorescences and leaves of ‘Robpatpas’.

### DETAILED BOTANICAL DESCRIPTION

In the following description, color references are made to the Royal Horticultural Society Colour Chart except where general terms of ordinary dictionary significance are used. Plants used for the photographs and description were planted in hanging basket containers after rooting and grown for about 3 months during the spring in a polyethylene-covered greenhouse in Grain Valley, Miss.. During the production of the plants, day temperatures were about 24° C. and night temperatures were about 18° C.

BOTANICAL CLASSIFICATION:

Lantana camara cultivar Robpatpas.

PARENTAGE:

Female parent.—Lantana camara cultivar Patriot Hot Country, disclosed in U.S. Plant Patent application serial No. 09/350,381.

Male parent.—Unidentified Lantana camara selection, not patented.

PROPAGATION:

Type cutting.—Terminal cuttings.

Time to initiate roots, summer.—About 13 days at 27° C.

Time to initiate roots, winter.—About 15 days at 27° C.

Time to develop roots, summer.—About 39 days at 29° C.

Time to develop roots, winter: About 46 days at 24° C.

Root description: Fine, fibrous; initially glaucous white in color then becoming tan, 161D, with development.

Rooting habit.—Freely branching.

PLANT DESCRIPTION:

Form.—Flowering subshrub; upright and outwardly spreading; mounded plant form. Freely branching; two lateral branches potentially forming at every node; pinching enhances lateral branch development.

Plant height.—About 22 cm.

Plant diameter.—About 48 cm.

Vigor.—Vigorous, rapid growth rate.

Lateral branches.—Length: About 18 cm. Diameter: About 3.5 mm. Internode length: About 4.2 cm. Strength: Flexible, but strong. Texture: Rough, pubescent. Color: Young: 144A. Woody: Closest to 165B. Foliage description: Leaves simple, generally symmetrical and long-persisting; opposite. Length: About 4.9 cm. Width: About 3.3 cm. Shape: Ovate. Apex: Acute. Base: Obtuse to truncate. Margin: Crenate to serrate with ciliation. Texture, both surfaces: Leathery, rough, coarse; slightly pubescent. Luster: Upper surface: Somewhat glossy. Lower surface: Dull. Venation pattern: Arcuate. Fragrance: Mint-like. Color: Young foliage, upper surface: More green than 146A. Young foliage, lower surface: More green than 146B. Mature foliage, upper surface: More green than 146A. Mature foliage, lower surface: More green than 147B. Venation, upper surface: 146A. Venation, lower surface: Close to 147C. Petiole length: About 8 mm. Petiole diameter: About 1.5 mm. Petiole texture, both surfaces: Slightly pubescent. Petiole color: Upper surface: 146A. Lower surface: Close to 146B.

FLOWER DESCRIPTION:

Flower type and habit.—Small salverform flowers arranged in axillary umbels; flowers face mostly upward or outward. Flowers self-cleaning. Very freely flowering with potentially two inflorescences per node; typically about flowers per umbel.

Natural flowering season.—Spring until frost in the autumn; flowering continuous and consistent.

Flower longevity on the plant.—About one week.

Fragrance.—Faint, spicy, pleasant.

Inflorescence diameter.—About

Inflorescence height.—About

Flowers.—Appearance: Flared trumpet, corolla fused, four-parted; flowers roughly rectangular in shape. Diameter: About Corolla tube length: About

Flower buds (showing color).—Length: About Diameter: About Shape: Elongate, oblong. Color:

Corolla.—Arrangement/appearance: Single whorl of four petals, fused into flared trumpet. Petal length from throat: About Petal width: Upper and lower petals: About Lateral petals: About Petal shape: Irregularly ovoid to almost orbicular. Petal apex: Rounded with slight emargination. Petal margin: Entire. Petal texture: Smooth, velvety. Color: Petal, upper surface, when opening: Petal, lower surface, when opening: Petal, upper surface, fully opened: Petal, lower surface and tube, fully opened:

Calyx.—Arrangement/appearance: One single calyx tube per flower. Calyx length: About Calyxwidth: About Apex: Broadly acute. Texture: Pubescent. Color, both surfaces:

Peduncles.—Length: About Diameter: About Angle: About 45° from the stem. Strength: Flexible, but strong. Texture: Color:

Pedicels.—Length: Less than Diameter: Less than Angle: About 45° from the stem. Strength: Flexible, but strong. Texture: Color:

Reproductive organs.—Stamens: Quantity/arrangement: Four per flower, adnate to floral tube. Filament length: Filament color: Anther shape: Ovoid. Anther length: About Anther color: Pollen amount: Pollen color: Pistils: Quantity: One per flower. Pistil length: About Stigma shape: Bilobed. Stigma color: Style color: About Style color: Ovary color:

Fruit.—Type: Drupe. Length: About Diameter: About Texture: Smooth. Color:

Seed.—Quantity per fruit: Two. Size: Color:

DISEASE/PEST RESISTANCE.—Plants of the new Lantana grown in the garden have been noted to be resistant to pathogens or pests common to Lantana. When grown in the greenhouse, plants of the new Lantana have been observed to be attractive to whiteflies.

WEATHER TOLERANCE: Plants of the new Lantana have been observed to be very tolerant to rain and wind.

TEMPERATURE TOLERANCE: Plants of the new Lantana have been observed to be tolerant to temperatures ranging from 0 to 38° C.

It is claimed:

1. A new and distinct cultivar of Lantana plant named ‘Robpatpas’, as illustrated and described.
